(19) United States
(12) Patent Application Publication
(10) Pub. No.: US 2017/0274068 A1
(43) Pub. Date: Sep. 28, 2017

(54) IMMUNOLOGICAL REAGENT

(71) Applicant: THE UNIVERSITY OF MELBOURNE, Victoria, (AU)

(72) Inventors: DAVID CHARLES JACKSON, VICTORIA (AU); BRENDON YEW LOONG CHUA, VICTORIA (AU); TOSHIKI SEKIYA, VICTORIA (AU)

(21) Appl. No.: 15/509,997

(22) PCT Filed: Sep. 11, 2015
(86) PCT No.: PCT/AU2015/050540

§ 371 Date: Mar. 9, 2017

(30) Foreign Application Priority Data
Sep. 12, 2014 (AU) 2014903652
Sep. 26, 2014 (AU) 2014903840

(51) Int. Cl. A61K 39/39; A61K 39/095; A61K 45/06; A61K 33/42

(52) U.S. Cl. CPC A61K 39/39; A61K 33/42; A61K 39/095; A61K 45/06; A61K 39/00

(57) ABSTRACT

The present invention provides an immunogenic composition comprising a charged antigen electrostatically associated with a Toll-Like Receptor (TLR) targeting moiety. The TLR targeting moiety comprises a TLR-2 agonist covalently attached to polyethylene glycol and to a hyper-branched charged peptide.

This application is associated with and claims priority from Australian patent application no. 2014903652 filed on 12 Sep. 2014 and Australian patent application no. 2014903840 filed on 26 Sep. 2014, the entire contents of each of these applications are incorporated herein by reference.

### FIELD OF THE INVENTION

The present invention relates to an immunological reagent for use on its own or in vaccine compositions and to methods of eliciting an immune response.

### BACKGROUND OF THE INVENTION

There is an ever increasing interest in the development of compositions which can be used to raise an immune response in humans and other animals in particular to protect against disease. In generating an adaptive immune response it is necessary for the antigen to encounter dendritic cells. Dendritic cells are immune cells and form part of the mammalian immune system. Their main function is to process antigen material and present it on the surface to other cells of the immune system, thus functioning as antigen-presenting cells as well as initiating innate immune responses.

The targeting of antigens to dendritic cells has been contemplated previously and lipopeptides which include lipids which Toll-like receptors on dendritic cells have been shown to elicit strong immune response (WO 2004/014956, WO 2004/014957, WO 2009/046498 & WO 2010/115230 the disclosures of which are incorporated herein by reference).

For any peptide to be able to induce an effective antibody response it must contain particular sequences of amino acids known as epitopes that are recognised by the immune system. In particular, for antibody responses, epitopes need to be recognised by specific immunoglobulin (Ig) receptors present on the surface of B lymphocytes. It is these cells which ultimately differentiate into plasma cells capable of producing antibody specific for that epitope.

In the case of viral infections and in many cases of cancer, antibody is of limited benefit in recovery and the immune system responds with cytotoxic T cells (CTL) which are able to kill the virus-infected or cancer cell. Like helper T cells, CTL are first activated by interaction with APC bearing their specific peptide epitope presented on the surface, this time in association with MHC class I rather than class II molecules. Once activated the CTL can engage a target cell bearing the same peptide/class I complex and cause its lysis.

### SUMMARY OF THE INVENTION

The present inventors have developed a novel immunogenic compositions which can elicit a heightened CD8⁺ response and/or a heightened antibody when the antigen in the composition is in a dose sparing amount.

Accordingly, in a first aspect the present invention provides an immunogenic composition comprising a charged antigen electrostatically associated with a Toll-Like Receptor (TLR) targeting moiety, wherein the TLR targeting moiety comprises a TLR-2 agonist covalently attached to polyethylene glycol and to a hyper-branched charged peptide.

In a second aspect the present invention provides a method of eliciting a CD8+ response in a subject, the method comprising administering to the subject the composition of the first aspect of the present invention.

In a third aspect the present invention provides the use of the composition of the first aspect of the present invention in the preparation of a medicament for use in eliciting a CD8⁺ response in a subject.

### BRIEF DESCRIPTION OF FIGURES

FIG. 1. Schematic representation of branched cationic Pam₂Cys-based lipopeptides. (A) R₄Pam₂Cys comprises of a branched structure mediated by a scaffold of lysine (Lys) residues to which 4 arginine (Arg) residues are attached to give an overall net N-terminal charge of +8. The lipid moiety Pam₂Cys is conjugated on the ε-amino group of the C-terminal Lys residue through 2 serine (Ser) residues. For the PEGylation of R₄Pam₂Cys, a glycine (Gly) residue was incorporated at the C-terminus of the structure followed by a 5, 11 or 22-unit polyethylene glycol (PEG) chain followed by the assembly of the branched structure and lipid to generate R₄Pam₂Cys-PEG₅, R₄Pam₂Cys-PEG₁₁ or R₄Pam₂Cys-PEG₂₂. All lipopeptides contain a carboxamide group (CONH₂) at the C-terminus.

FIG. 2. Optical density of antigen and lipopeptide formulations. A constant amount (0.55 nmol; 25 μg) of OVA was mixed with each lipopeptide at different protein:lipopeptide molar ratios in 50 μl of PBS in a 96-well plate at room temperature. The optical density of these solutions was then measured on a plate reader at a wavelength of 450 nm.

FIG. 3. Particle size distribution of antigen and lipopeptide formulations. OVA (25 μg) was mixed with each lipopeptide at a 1:3 molar ratio of protein to lipopeptide in 50 50 μl of PBS in a 96-well plate at room temperature. The size distribution of particulates in samples were then analysed by dynamic light scattering with each profile depicting the hydrodynamic radius (nm) of particles detected versus the percentage intensity of scattered light (% intensity).

FIG. 4. Antibody responses following vaccination with antigen associated with R₄Pam₂Cys or PEGylated R₄Pam₂Cys. BALB/c mice (n=5/group) were inoculated via the subcutaneous route with 25, 12.5, 6.25, or 3.12 μg of OVA formulated with R₄Pam₂Cys or R₄Pam₂Cys-PEG₁₁ at a 1:5 molar ratio of protein to lipopeptide on day 0 and 21. Sera were obtained 14 days following the second inoculation. Antibody titres were then determined by ELISA using OVA as the coating antigen. Titres from individual animals are presented with the horizontal line and error bars representing the mean value and standard deviation of each group. A two-way ANOVA analysis of variance followed by a Bonferroni post-hoc range test was used to calculate P-values where *P<0.01, **P<0.001 and ***P<0.0001.

FIG. 5. CD8⁺ T cell responses following vaccination with antigen associated with R₄Pam₂Cys or PEGylated R₄Pam₂Cys. C57BL/6 (n=3/group) mice were inoculated via the subcutaneous route with various amounts of OVA alone or in the presence of each lipopeptide at a 1:3 molar ratio. Spleens were obtained 10 days after immunisation and OVA₂₅₇ specific IFN-γ secreting CD8⁺ T cells were enumerated by intracellular cytokine staining assay (ICS). P-values were measured using an ANOVA two-way analysis of variance followed by a Bonferroni post-hoc range test where *P<0.01, **P<0.001, ***P<0.0001, #P<0.05.

FIG. 6. In vivo proliferative T cell responses. (A) To measure in vivo antigen presentation leading to T cell activation, CFSE labelled CD8⁺CD45.1⁺ OT-I cells (10⁶ cells) were transferred intravenously into naïve C57BL/6 mice (n=3/group) and vaccinated the next day via the subcutaneous route with 3 μg of OVA alone or OVA formulated with R₄Pam₂Cys or with R₄Pam₂Cys-PEG₁₁ at a 1:3 molar ratio. Inguinal LNs were obtained 3 days after vaccination and CFSE intensities of CD8⁺CD45.1⁺ cells measured by flow cytometry. Figures within each histogram represent the percentage (±SD) of cells which proliferated as defined by their lower CFSE expression. (B) Also presented is the total number (±SD) of OT-I cells that had undergone more than one round of division detected in both inguinal LNs of each animal. The asterisk (*) indicates P values <0.05 when analysis of variance was analysed in a one-way ANOVA followed by a Tukey test.

FIG. 7. Lung viral titres and recall CD8⁺ T cell responses in virus challenged and vaccinated mice. Groups of C57BL/6J mice (n=5) were inoculated via the intranasal route with 25 μg of OVA in the presence of R₄Pam₂Cys-PEG₁₁ or R₄Pam₂Cys at a 1:3 molar ratio of protein to lipopeptide. Mice were challenged intranasally 28 days later with 10^{4.5} pfu of A/HK×31 influenza virus containing H-2K^{b}OVA₂₅₇ epitope (X31-OVA). Lungs were obtained at 3 days post-viral challenge and viral titres in the lung homogenates were determined using a standard MDCK plaque assay (A). OVA₂₅₇ specific IFN-γ production by CD8⁺ T cells was also enumerated by intracellular cytokine staining assay (B).

FIG. 8. Vaccine-mediated protection against melanoma tumor challenge. Male C57BL/6 mice (n=5/group) were inoculated via the subcutaneous route with saline, 25 μg of OVA alone or with R₄Pam₂Cys or R₄Pam₂Cys-PEG₁₁ at a 1:3 molar ratio. All mice were challenged with 10⁴ B16.mOVA cells 7 days following the last inoculation and monitored for tumour growth. Mice were euthanized when tumour volume exceeded 700 mm³. A log-rank (Mantel-Cox) test was used to analyse the variance between the indicated groups and the asterisk (*) indicates P values<0.05.

FIG. 9. Schematics of the K4, K8, R4, R8, E4, E8, D4 and D8 structures of the TLR targeting moiety of the present invention. The PEG₁₁ species only is shown. It is understood that the PEG_{S} and PEG₂₂ species have the same basic structure.

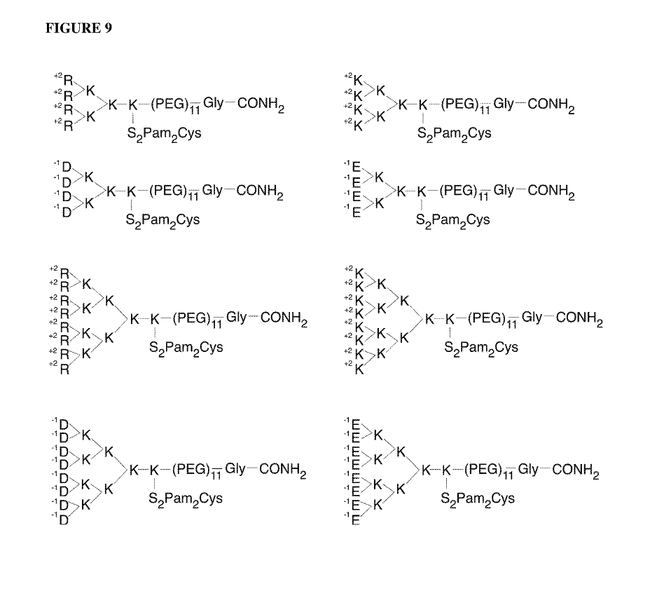

### DETAILED DESCRIPTION OF THE INVENTION

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

All publications mentioned in this specification are herein incorporated by reference in their entirety.

It must be noted that, as used in the subject specification, the singular forms “a”, “an” and “the” include plural aspects unless the context clearly dictates otherwise. Thus, for example, reference to “an agent” includes a single agent, as well as two or more agents; reference to “the composition” includes a single composition, as well as two or more compositions; and so forth.

In this specification the term “TLR2” is intended to mean Toll-Like Receptor 2 protein. TLR2 is a membrane receptor protein family of Toll-Like Receptors (i.e. “TLRs”) including TLR1, TLR3, TLR4, TLR5, TLR6, TLR7, TLR8 and TLR9. In humans, TLR2 is encoded by the TLR2 gene. TLR2 is expressed on the surface of certain cells and plays a fundamental role in pathogen recognition and activation of innate immunity.

A TLR2 agonist is an agent that binds Toll-like receptor 2. The TLR2 agonist may bind TLR2 as a homodimer or heterodimer.

The present invention is based on the surprising finding that a significantly enhanced CD8⁺ response is achieved using a composition comprising a charged antigen comprising a CTL epitope electrostatically associated with a TLR2 agonist such as S-[2,3-bis(palmitoyloxy)propyl]cysteine if polyethylene glycol is covalently attached to the TLR2 agonist. Further the present inventors have also found that a heightened antibody response can be obtained when the antigen in the composition is in a dose sparing amount.

Accordingly, in a first aspect the present invention provides an immunogenic composition comprising a charged antigen electrostatically associated with a Toll-Like Receptor (TLR) targeting moiety, wherein the TLR targeting moiety comprises a TLR-2 agonist covalently attached to polyethylene glycol and to a hyper-branched charged peptide.

In a second aspect the present invention provides a method of eliciting a CD8⁺ response in a subject, the method comprising administering to the subject the composition of the first aspect of the present invention.

The present inventors have developed novel immunogenic compositions which can elicit a heightened CD8⁺ response and/or a heightened antibody when the antigen in the composition is in a dose sparing amount.

In a third aspect the present invention provides the use of the composition of the first aspect of the present invention in the preparation of a medicament for use in eliciting a CD8⁺ response in a subject.

In a preferred embodiment of the present invention the charged antigen comprises a cytotoxic T-cell (CTL) epitope.

In another preferred embodiment of the present invention the charged antigen comprises a B-cell epitope and the antigen is present in the composition. In another preferred embodiment of the present invention, the charged antigen comprises a B-cell epitope and the antigen is present in the composition in a dose sparing amount.

In some embodiments, the TLR2 agonist is a lipopeptide or comprises a lipid moiety.

An exemplary lipopeptide in accordance with this embodiment of the present invention is the lipopeptide “Pam₂Cys”. One of skill in the art would understand that the term “lipopeptide” means any composition of matter comprising one or more lipid moieties and one or more amino acid sequences that are conjugated. “Pam₂Cys” (also known as dipalmitoyl-S-glyceryl-cysteine or S-[2, 3 bis(palmitoyloxy) propyl] cysteine has been synthesised and corresponds to the lipid moiety of MALP-2, a macrophage-activating lipopeptide isolated from Mycoplasma fermentans. Pam₂Cys is known to be a ligand of TLR2.

Pam₂Cys has the structure:

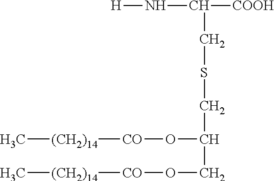

Another exemplary lipopeptide is the lipoamino acid N-palmitoyl-S-[2, 3-bis (palmitoyloxy) propyl] cysteine, also known as Pam₃Cys or Pam₃Cys-OH, is a synthetic version of the N-terminal moiety of Braun's lipoprotein that spans the inner and outer membranes of Gram negative bacteria. Pam3Cys has the following structure:

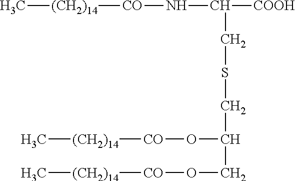

U.S. Pat. No. 5,700,910 describes several N-acyl-S-(2-hydroxyalkyl) cysteines for use as intermediates in the preparation of lipopeptides that are used as synthetic adjuvants, B lymphocyte stimulants, macrophage stimulants, or synthetic vaccines. U.S. Pat. No. 5,700,910 also teaches the use of such compounds as intermediates in the synthesis of Pam3Cys-OH and of lipopeptides that comprise this lipoamino acid or an analog thereof at the N-terminus.

Other lipid moieties which may be used to target cell surface TLRs include palmitoyl, myristoyl, stearoyl, lauroyl, octanoyl, or decanoyl.

In addition to Pam2Cys and Pam3Cys, the present invention also contemplates the use of Ste2Cys, Lau2Cys and Oct2Cys according to the present invention. Those skilled in the art will be aware that Ste2Cys is also known as S-[2, 3-bis (stearoyloxy) propyl] cysteine or distearoyl-S-glyceryl-cysteine; that Lau2Cys is also known as S-[2, 3-bis (lauroyloxy) propyl] cysteine or dilauroyl-S-glyceryl-cysteine); and that Oct2Cys is also known as S-[2,3-bis (octanoyloxy) propyl] cysteine or dioctanoyl-S-glyceryl-cysteine).

Other suitable TLR2 agonists include, but are not limited to, synthetic triacylated and diacylated lipopeptides, FSL-I (a synthetic lipoprotein derived from Mycoplasma salivarium I), Pam₃Cys (tripalmitoyl-S-glyceryl cysteine) and S-[2,3-bis(palmitoyloxy)-(2RS)-propyl]-N-palmitoyl-(R)-cysteine, where “Pam₃” is “tripalmitoyl-S-glyceryl”. Derivatives of Pam₃Cys are also suitable TLR2 agonists, where derivatives include, but are not limited to, S-[2,3-bis(palmitoyloxy)-(2-R,S)-propyl]-N-palmitoyl-(R)-Cys-(S)-Ser-(Lys)₄-hydroxytrihydrochloride; Pam₃Cys-Ser-Ser-Asn-Ala; PaM₃Cys-Ser-(Lys)₄; Pam₃Cys-Ala-Gly; Pam₃Cys-Ser-Gly; Pam₃Cys-Ser; PaM₃CyS-OMe; Pam₃Cys-OH; PamCAG, palmitoyl-Cys((RS)-2,3-di(palmitoyloxy)-propyl)-Ala-Gly-OH; and the like. Another non-limiting examples of suitable TLR2 agonists are Pam₂CSK₄ (dipalmitoyl-S-glyceryl cysteine-serine-(lysine)₄; or Pam₂Cys-Ser-(Lys)₄) is a synthetic diacylated lipopeptide. Other synthetic TLRs agonists include those described, e.g., in Kellner et al. (1992, Biol. Chem. 373:1 :51-5); Seifer et al. (1990, Biochem. J, 26:795-802) and Lee et al. (2003, J. Lipid Res., 44:479-486).

In the immunogenic composition of the present invention the antigen comprising the CTL epitope is electrostatically associated with the TLR2 agonist due to the charge provided by the hyper-branched peptide. Depending on the charge of the antigen the hyper-branched peptide may have either a positive or negative charge.

A hyper-branched peptide is a polymer that comprises a dendrite structure terminating in a plurality of amino acids; for example, terminating in at least four amino acids, in at least eight amino acids, and so on. A hyper-branched peptide may comprise a regularly ordered branch structure and/or an irregular branch structure. Examples of hyper-branched charged peptides are provided in WO 2009/046498, WO 2010/115230 and WO 2012/037612. The hyper-branched peptide comprises a dendrite structure terminated in amino acids of the desired charge. In preferred forms this dendrite structure is composed of lysine residues where further residues are attached to both the a and c amino groups. This is shown schematically below.

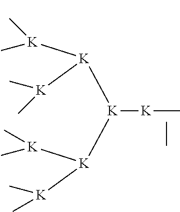

Whilst the examples of the current application involve branched poly-lysine structures person skilled in the art will readily recognize that other dendrite structures may be used.

Where it is desired that that the hyper-branched peptide has a positive charge it is preferred that the peptide is terminated with at least 4 positively charged amino acids. This may be, for example, 4 arginine residues or 4 lysine residues or combinations thereof. Preferred forms include R4, R8, K4, K8, H4, and H8.

Where it is desired that that the hyper-branched peptide has a negative charge it is preferred that the peptide is terminated with at least 4 negatively charged amino acids. This may be, for example, 4 glutamate residues or 4 aspartate residues or combinations thereof. Preferred forms include E4, E8, D4, and D8. Illustrations of these structures are provided in FIG. 13.

As would be readily understood polyethylene glycol (PEG) is an ethylene oxide polymer comprising repeating ethylene oxide subunits. In preferred embodiments of the present invention the PEG linked to the TLR2 agonist has 5 to 22, preferably 8 to 15, more preferably 9 to 13 ethylene oxide subunits. It is currently preferred that the PEG has 10 to 12 subunits preferably 11.

The CTL epitope is conveniently derived from the amino acid sequence of an immunogenic protein, lipoprotein, or glycoprotein of a virus, prokaryotic or eukaryotic organism, including but not limited to a CTL epitope derived from a mammalian subject or a bacterium, fungus, protozoan, or parasite that infects said subject.

The CTL epitope will be capable of eliciting a T cell response when administered to a, mammal, preferably by activating CD8+ T cells specific for the epitope or antigen from which the epitope was derived, and more preferably, by inducing cell mediated immunity against the pathogen or tumour cell from which the epitope is derived.

Preferred CTL epitopes from parasites are those associated with leishmania, malaria, trypanosomiasis, babesiosis, or schistosomiasis, such as, for example a CTL epitope of an antigen of a parasite selected from the group consisting of Plasmodium falciparum, Circumsporozoa, Leishmania donovani, Toxoplasma gondii, Schistosoma mansoni, Schistosoma japonicum, Schistosoma hematobium and Trypanosomabrucei.

Preferred virus-specific CTL epitopes are derived from Rotaviruses, Herpes viruses, Corona viruses, Picornaviruses (eg. Apthovirus), Respiratory Synctial virus, Influenza Virus, Parainfluenza virus, Adenovirus, Pox viruses, Bovine herpes virus Type I, Bovine viral diarrhea virus, Bovine rotaviruses, Canine Distemper Virus (CDV), Foot and Mouth Disease Virus (FMDV), Measles Virus, Human Immunodeficiency Viruses (HIV), Feline Immunodeficiency Viruses (FIV), Epstein-Barr virus (EBV), Human Cytomegalovirus (HCMV), or hepatitis viruses, in particular HCV, and the like.

Preferred bacteria-specific CTL epitopes are derived from Pasteurella, Actinobacillus, Haemophilus, Listeria monocytogenes, Mycobacterium tuberculosis, Staphylococcus, Neisseria gonorrhoeae, Helicobacter pylori, Streptococcus pneumoniae, Salmonella enterica, E. coli, Shigella, and the like.

Preferred CTL epitopes from mammalian subjects are derived from and/or capable of generating T cell responses against a tumor CTL antigen. Tumour specific CTL epitopes are usually native or foreign CTL epitopes, the expression of which is correlated with the development, growth, presence or recurrence of a tumor. In as much as such CTL epitopes are useful in differentiating abnormal from normal tissue, they are useful as a target for therapeutic intervention. Such CTL epitopes are well known in the art. Indeed, several examples are well characterized and are currently the focus of great interest in the generation of tumor-specific therapies. Non-limiting examples of tumor CTL epitopes are derived from carcinoembryonic antigen (CEA), prostate specific antigen (PSA), melanoma antigen (MAGE, BAGE, GAGE), and mucins, such as MUC-1.

International Patent application no. WO 2004/014957, the disclosure of which is incorporated by cross reference, provides a useful disclosure of various CTL epitopes.

The term “subject” as used herein refers to an animal, in particular a mammal and more particularly a primate including a lower primate and even more particularly, a human who can benefit from the medical protocol of the present invention. A subject regardless of whether a human or non-human animal or embryo may be referred to as an individual, subject, animal, patient, host or recipient. The present invention has both human and veterinary applications. For convenience, an “animal” specifically includes livestock animals such as cattle, horses, sheep, pigs, camelids, goats and donkeys. With respect to horses, these include horses used in the racing industry as well as those used recreationally or in the livestock industry. Examples of laboratory test animals include mice, rats, rabbits, guinea pigs and hamsters. Rabbits and rodent animals, such as rats and mice, provide a convenient test system or animal model as do primates and lower primates. In some embodiments, the subject is human.

The composition according to the present invention is to be administered in an effective amount. The terms “effective amount” and “therapeutically effective amount” of a TLR2 moiety, as used herein, mean a sufficient amount to provide in the course the desired therapeutic or physiological effect in at least a statistically significant number of subjects. Undesirable effects, e.g. side effects, are sometimes manifested along with the desired therapeutic effect; hence, a practitioner balances the potential benefits against the potential risks in determining what is an appropriate “effective amount”. The exact amount required will vary from subject to subject, depending on the species, age and general condition of the subject, mode of administration and the like. Thus, it may not be possible to specify an exact “effective amount”. However, an appropriate “effective amount” in any individual case may be determined by one of ordinary skill in the art using only routine experimentation. In some embodiments, an effective amount for a human subject lies in the range of about 0.1 nmol/kg body weight/dose to 1 mol/kg body weight/dose. In some embodiments, the range is about 1 nmol to 1 mol, about 1 μmol to 1 mol, 1 μmol to 500 μmol, 1 μmol to 250 μmol, 1 μmol to 50 μmol, or 1 nmol to 1 μmol/kg body weight/dose. In some embodiments, the range is about 0.08 μmol to 0.11 μmol/kg body weight/dose of the TLR2 moiety. Dosage regimes are adjusted to suit the exigencies of the situation and may be adjusted to produce the optimum therapeutic dose. For example, several doses may be provided daily, weekly, monthly or other appropriate time intervals.

The terms “treatment” or “treating” include, but are not limited to, (i) slowing or arresting the progression of disease, (ii) partially reversing the progression of disease and (iii) completely reversing the progression of disease (i.e., curing the disease). The terms “prevent” or “preventing” should not be construed as being limited to the complete prevention of disease (i.e., causing the disease not to develop), but may include minimizing the progression of disease, for example, where the disease occurs with less intensity or progresses at a slower rate in a subject as a result of the prophylactic administration of the composition according to the present invention.

The composition according to the invention may be administered in a single dose or a series of doses. While it is possible for the conjugate to be administered alone, it is preferable to present it as a composition, preferably as a pharmaceutically composition. The formulation of such compositions is well known to those skilled in the art. The composition may contain any pharmaceutically acceptable carriers, diluents or excipients. Suitable dosage amounts and dosing regimens can be determined by the attending physician and may depend on the particular condition being treated, the severity of the condition as well as the general age, health and weight of the subject.

By “pharmaceutically acceptable” carrier, excipient or diluent is meant a pharmaceutical vehicle comprised of a material that is not biologically or otherwise undesirable, i.e. the material may be administered to a subject along with the selected conjugate without causing any or a substantial adverse reaction. Carriers may include excipients and other additives such as diluents, detergents, colouring agents, wetting or emulsifying agents, pH buffering agents, preservatives, and the like. Carriers may also include all conventional solvents, dispersion media, fillers, solid carriers, coatings, antifungal and antibacterial agents, dermal penetration agents, surfactants, isotonic and absorption agents and the like. It will be understood that the compositions of the invention may also include other supplementary physiologically active agents.

The compositions of the present invention may be administered by any means known to those skilled in the art, including, but not limited to, intranasally, orally, subcutaneously, intramuscularly and intravenously.

Those skilled in the art will appreciate that the invention described herein in susceptible to variations and modifications other than those specifically described. It is to be understood that the invention includes all such variations and modifications which fall within the spirit and scope. The invention also includes all of the steps, features, compositions and compounds referred to or indicated in this specification, individually or collectively, and any and all combinations of any two or more of said steps or features.

### EXAMPLES

### Example 1

### PEGylation of R₄Pam₂Cys Results in the Formation of Smaller Sized Particles and Better Solubility when Associated with Antigen

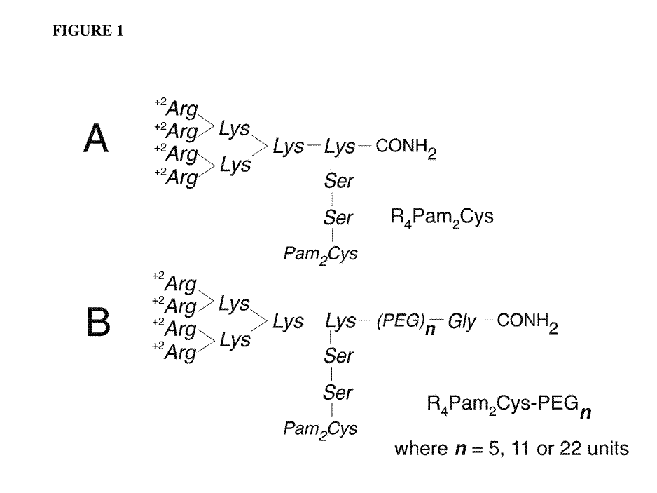

To facilitate association of Pam₂Cys with protein antigens, the lipid moiety was assembled as a branched cationic structure, R₄Pam₂Cys, containing 4 N-terminal arginine residues designed to bind electrostatically to oppositely charged regions on an antigen (FIG. 1A). To determine the effect of PEGylation on R₄Pam₂Cys, we incorporated a PEG molecule at the C terminus of R₄Pam₂Cys leaving the N-terminal R residues free to electrostatically bind to oppositely charged regions of an antigen (FIG. 1B). PEG lengths of 5, 11 and 22 units were incorporated to produce R₄Pam₂Cys-PEG₅, R₄Pam₂Cys-PEG₁₁ and R₄Pam₂Cys-PEG₂₂ respectively. All synthesised lipopeptides presented as a single peak when analysed by HPLC and exhibited the correct molecular weight.

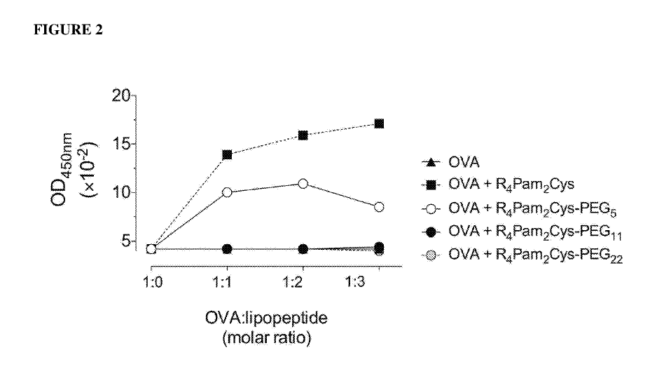

We have previously found that electrostatic association between OVA and R₄Pam₂Cys results in the formation of macromolecular complexes that can be measured by determining the optical density (OD) of solutions containing these constituents (Chua 2011). Adding increasing amounts of R₄Pam₂Cys to a constant amount of OVA results in an increase in OD (FIG. 2). This effect was reduced when R₄Pam₂Cys-PEG₅ was used with lower OD readings detected in all samples containing this lipopeptide and antigen. In samples containing antigen in the presence of R₄Pam₂Cys-PEG₁₁ or R₄Pam₂Cys-PEG₂₂, however, the OD and hence their solubility remained similar to solutions that contained OVA alone despite up to a 3-fold molar excess of each lipopeptide being used.

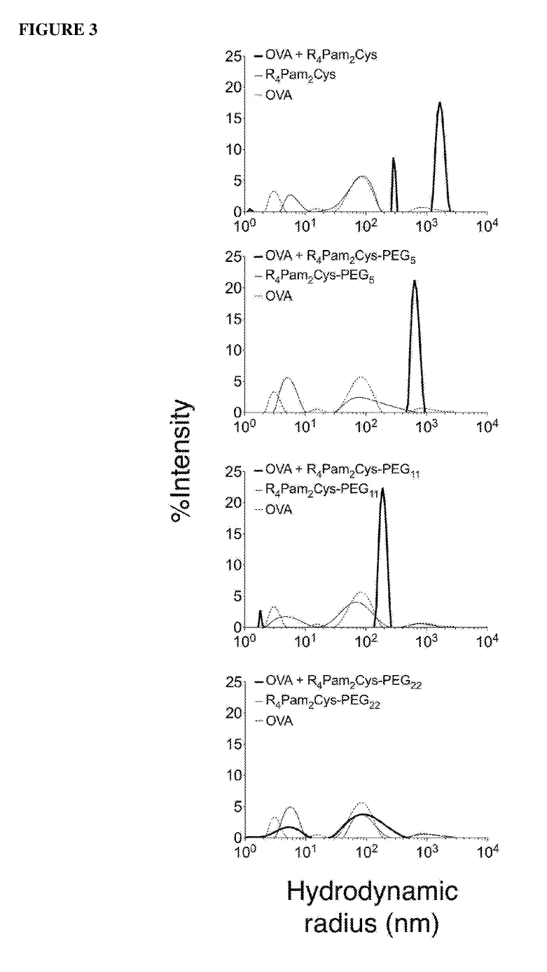

To determine the size of complexes formed between OVA and each lipopeptide, we analysed these formulations by dynamic light scattering. Our results (FIG. 3, Table 1) showed that solutions containing OVA or each of the lipopeptides alone present as particles of ˜145-275 nm in diameter. Association of OVA and R₄Pam₂Cys resulted in the formation of larger sized complexes with the majority being ˜3122 nm in size and a small minority presenting as ˜638 nm. The average size of complexes formed using PEGylated lipopeptides were, however, smaller in comparison and exhibited a hierarchy that was inversely proportional to the number of incorporated PEG units. Of the PEGylated lipopeptides examined, the largest antigen-lipopeptide complexes were formed with the use of R₄Pam₂Cys-PEG₅, which presented as particles of ˜1351 nm. This was not unexpected considering solutions containing these constituents exhibited the highest OD and least solubility (FIG. 2). In comparison, the size of complexes formed using R₄Pam₂Cys-PEG₁₁ was smaller with a significant proportion at ˜407 nm. The percentage polydispersity of complexes formed using either R₄Pam₂Cys-PEG₅ or R₄Pam₂Cys-PEG₁₁ were well within the range that would be considered monodispersed (Khurshid 2014) and indicative of their largely consistent and uniform size.

The smallest complexes formed were demonstrated using R₄Pam₂Cys-PEG₂₂ at ˜224 nm and exhibited a size range (215-234 nm) that was very close to or overlapped with those exhibited by the lipopeptide (188-206 nm) or OVA (157-242 nm) alone. In fact, the size similarity of these complexes and its constituents was also reflected in their size distribution profiles and % polydispersity ranges suggesting little association between R₄Pam₂Cys-PEG₂₂ and OVA.

TABLE 1
Dynamic light scattering analysis of vaccine formulations*

| | Hydrodynamic | |
|---|---|---|
| Sample | diameter (nm) | % Pd^{a}\hat{ } |
| OVA | 199.5 ± 42.6 | 27.1 ± 11.5 |
| R₄Pam₂Cys | 149.2 ± 20.3 | 42.3 ± 17.8 |
| R₄Pam₂Cys-PEG₅ | 275.5 ± 4.8 | 71.0 ± 7.6 |
| R₄Pam₂Cys-PEG₁₁ | 145.3 ± 11.5 | 31.1 ± 27.6 |
| R₄Pam₂Cys-PEG₂₂ | 196.8 ± 9.0 | 40.5 ± 4.2 |
| OVA + R₄Pam₂Cys | 638.8 ± 55.9 | 10.4 ± 5.6 |
| | 3122.9 ± 295.1 | 5.5 ± 1.4 |
| OVA + R₄Pam₂Cys-PEG₅ | 1351.2 ± 61.2 | 11.4 ± 3.2 |
| OVA + R₄Pam₂Cys-PEG₁₁ | 407.6 ± 51.3 | 12.9 ± 3.0 |
| OVA + R₄Pam₂Cys-PEG₂₂ | 224.2 ± 9.6 | 48.7 ± 11.6 |

*Results were derived from measurements from duplicate samples over 3 separate experiments. Each measurement consists of 30 readings performed at 25° C.
\hat{ }Pd^{a} = polydispersity. A peak with <20% polydispersity is considered to be monodispersed (Khurshid 2014).

### Example 2

### Antibody Responses Induced Using R₄Pam₂Cys-PEG_{11H} with Dose Sparing Amounts of Antigen are Higher than Antibody Responses Obtained Using R₄Pam₂Cys

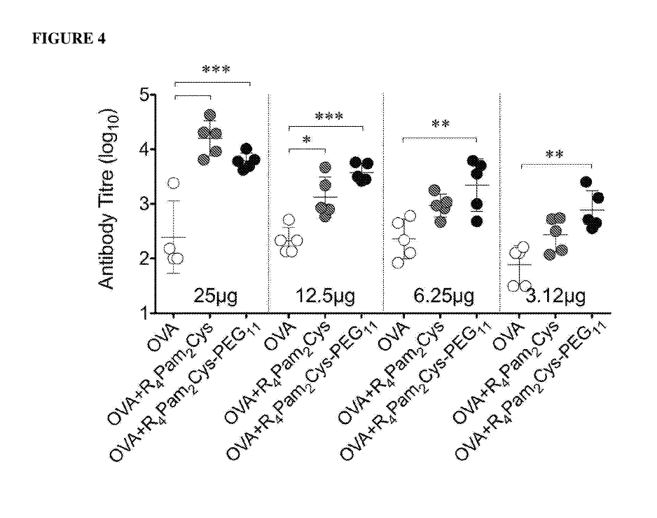

To compare the ability of antigens mixed with R₄Pam₂Cys or R₄Pam₂Cys-PEG₁₁ to induce antibody responses groups of male BALB/c mice (n=5) were inoculated via the subcutaneous route with 25, 12.5, 6.25, or 3.12 μg of OVA pre-mixed with R₄Pam₂Cys-PEG or R₄Pam₂Cys lipopeptide at a 1:5 molar ratio of protein to lipopeptide on day 0 and 21. Sera were obtained 14 days following the second inoculation. Antibody titres were then determined by ELISA using OVA as the coating antigen and the results are shown in FIG. 4.

Both lipopeptides were similar in their ability to induce higher antibody titres using 12.5 μg and 25 μg of OVA when compared to vaccinations performed using equivalent amounts of antigen alone (FIG. 4). At lower antigen doses of 6.25 μg and 3.12 μg, significantly higher titres were observed when formulated with R₄Pam₂Cys-PEG₁₁ indicating that PEGylation of this vaccine delivery vehicle improves its ability to induce dose-sparing antibody responses compared to antigen alone or antigen formulated with R₄Pam₂Cys.

### Example 3

### Better Endogenous Primary CD8⁺ T Cell Responses are Induced by Antigen Associated with R₄Pam₂Cys-PEG₁₁

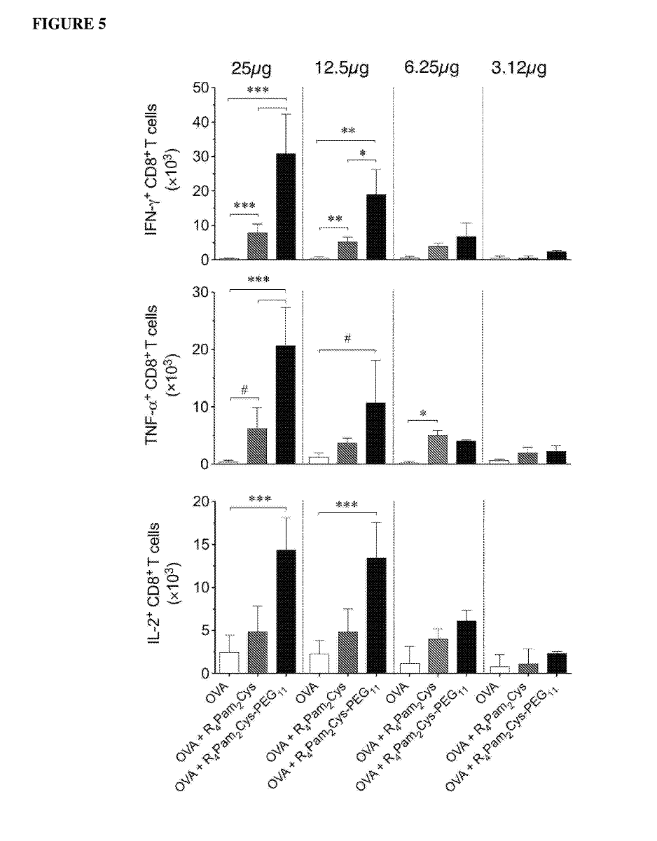

To determine the effect of PEGylation on the ability of R₄Pam₂Cys to mediate the induction of primary CD8⁺ T cell responses, C57BL/6 mice were vaccinated with various amounts of OVA alone or OVA in the presence of R₄Pam₂Cys or R₄Pam₂Cys-PEG₁₁. Splenocytes were obtained 10 days later and the presence of antigen-specific cytokine secreting CD8⁺ T cells were determined by intracellular cytokine staining (ICS) after in vitro re-stimulation with OVA257 peptide and analysis by flow cytometry. Vaccination of mice with 12.5-25 μg of OVA formulated with R₄Pam₂Cys resulted in significant increases in the number of cytokine-producing CD8⁺ T cells, in particular those producing IFN-γ and TNF-α, compared to vaccination with OVA alone (FIG. 5) which is in line with results from our previous studies (Chua 2011, Chua 2014). Responses obtained with equivalent amounts of OVA formulated with R₄Pam₂Cys-PEG₁₁, however, were not only higher compared to R₄Pam₂Cys but also induced significantly more IL-2 producing cells. These results indicate that the presence of PEG₁₁ within the structure of R₄Pam₂Cys enhances its ability to induce stronger cell-mediated responses.

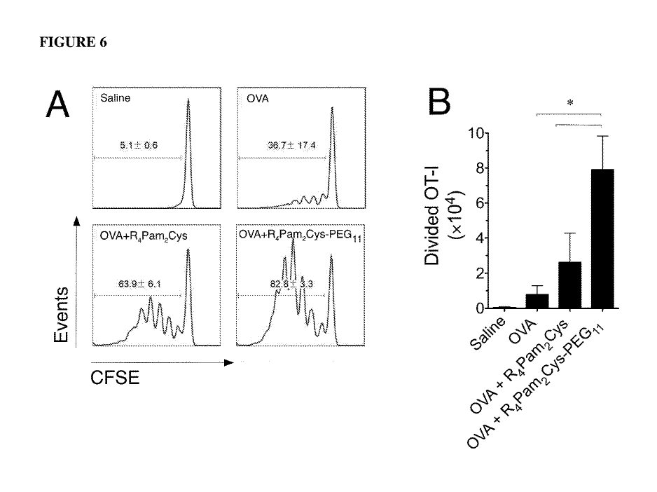

To characterise early events associated with vaccine-mediated induction of CD8⁺ T cell responses, the present inventors utilised the adoptive transfer of CD8⁺ T cells from OTI transgenic mice that express the Vα2 TCR specific for OVA₂₅₇. CFSE-labelled lymphocytes from OTI mice were transferred intravenously to C57BL/6 mice one day before subcutaneous inoculation with 3 μg of OVA alone or OVA formulated with a 3-fold molar excess of R₄Pam₂Cys or R₄Pam₂Cys-PEG₁₁. Analysis of OTI T cells in the draining lymph nodes of vaccinated animals was then carried out 3 days later and the loss of CFSE in these cells was used as an indicator of T cell proliferation. Vaccination with OVA was able to induce the division of T cells (˜37%) and it was clear that using equivalent amounts of antigen formulated with R₄Pam₂Cys resulted in significantly higher levels of T cell proliferation (p<0.01) with ˜1.5-fold more T cells observed (˜64%) (FIG. 6A). Proliferative responses however resulting from vaccinations carried out with OVA with R₄Pam₂Cys-PEG resulted in the division of ˜83% of T cells. This trend in results was also observed when absolute cell numbers in the nodes from each animal were taken into account wherein formulation of OVA with R₄Pam₂Cys resulted in ˜2-fold increase in T cell frequencies compared to OVA alone and up to nearly a 4-fold difference (p<0.05) observed with R₄Pam₂Cys-PEG (FIG. 6B).

### Example 4

### Vaccination of Antigen with R₄Pam₂Cys-PEG₁₁ Results in Better Anti-Viral Protection

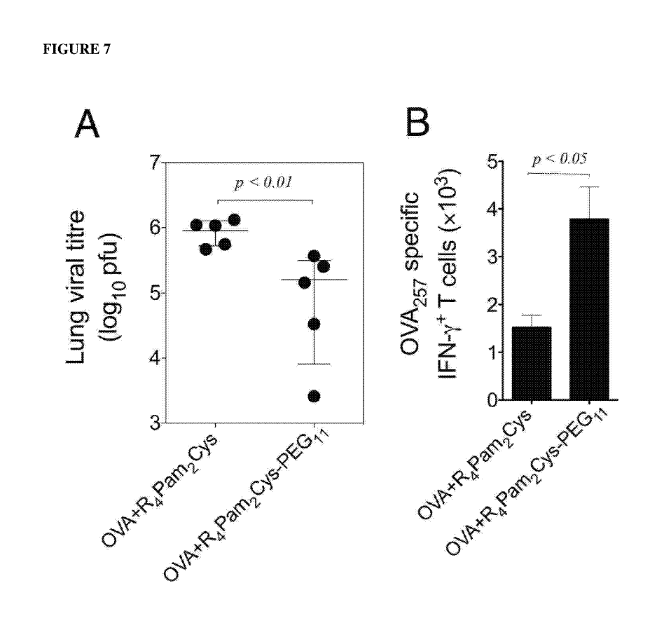

We hypothesised that the enhancement T cell responses in mice inoculated with PEGylated R₄Pam₂Cys may be reflected in better protection against viral pathogenic challenge. Therefore mice were inoculated via intranasal route with OVA formulated with R₄Pam₂Cys-PEG₁₁ or R₄Pam₂Cys and challenged 28 days later with a chimeric A/HK×31 influenza virus containing the H-2K^{b}OVA₂₅₇ epitope (X31-OVA). Lungs were harvested from mice 3 days after challenge and lung viral titres and number of OVA₂₅₇-specific IFN-γ secreting CD8⁺ T cells measured. The results show that mice inoculated with OVA+R₄Pam₂Cys-PEG₁₁ had significantly less virus in their lungs at day 3 compared to those inoculated with OVA+R₄Pam₂Cys (FIG. 7A). Enhanced clearance of influenza infection mediated by OVA+R₄Pam₂Cys vaccination has been previously shown at day 5 post-infection (Chua 2011), these results therefore indicate that faster clearance of infection can result from the use of R₄Pam₂Cys-PEG₁₁

Subsequently, when number of OVA₂₅₇ specific IFN-γ secreting CD8⁺ T cells in the lung was determined, ˜two-fold higher antigen-specific CD8⁺ T cells were detected in mice vaccinated with OVA-R₄Pam₂Cys-PEG₁₁ compared to R₄Pam₂Cys (FIG. 7B) leading us to conclude that the enhanced CD8⁺ T cell responses induced by OVA+R₄Pam₂Cys-PEG₁₁ leads to better protection against virus challenge.

### Example 5

### Vaccination of Antigen with R₄Pam₂Cys-PEG₁₁ Results in Better Anti-Viral Protection

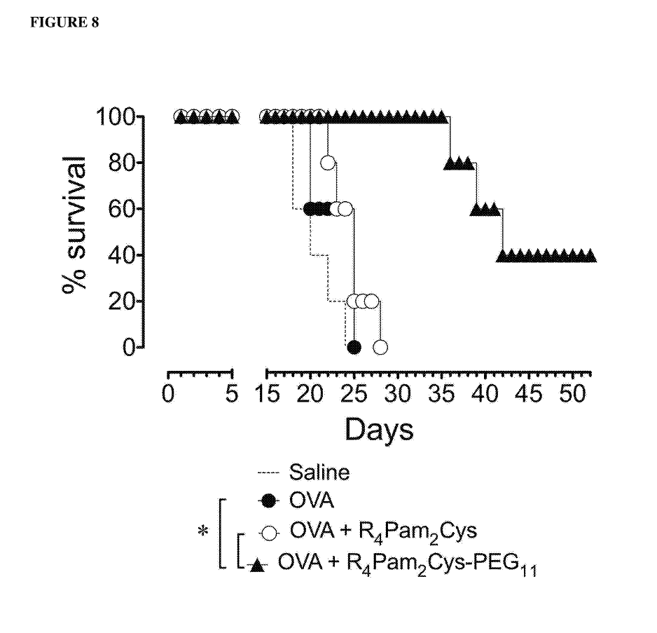

We also hypothesised that these enhanced CD8⁺ T cell responses can be beneficial for preventing the development of tumours. To test this, mice were vaccinated and challenged 7 days later with B16 melanoma cells containing membrane-bound OVA (B16.mOVA) and the progression of tumour growth and survival monitored. Compared to mice inoculated with saline or OVA alone, all of which succumbed to disease as a result of tumour formation within 25 days of challenge, an improvement in survival rate was observed following OVA inoculation using R₄Pam₂Cys (FIG. 8). All mice inoculated with OVA formulated with R₄Pam₂Cys-PEG survived significantly longer compared to other groups with 40% remaining after 50 days.

### REFERENCES

Chua B Y et al. Soluble proteins induce strong CD8+ T cell and antibody responses through electrostatic association with simple cationic or anionic lipopeptides that target TLR2. Journal of Immunology. 2011; 187:1692-701.
Chua B Y et al. Hepatitis C VLPs delivered to dendritic cells by a TLR2 targeting lipopeptide results in enhanced antibody and cell-mediated responses. PloS One. 2012; 7:e47492.
Chua B Y et al. The use of a TLR2 agonist-based adjuvant for enhancing effector and memory CD8 T-cell responses. Immunology and Cell Biology. 2014; 92:377-83.
Khurshid S et al. Porous nucleating agents for protein crystallization. Nature Protocols. 2014 July; 9 (7): 1621-33

We claim:

1. An immunogenic composition comprising a charged antigen electrostatically associated with a Toll-Like Receptor (TLR) targeting moiety, wherein the TLR targeting moiety comprises a TLR-2 agonist covalently attached to polyethylene glycol and to a hyper-branched charged peptide.

2. The composition according to claim 1, wherein the charged antigen comprises a cytotoxic T-cell (CTL) epitope.

3. The composition according to claim 1, wherein the charged antigen comprises a B-cell epitope and the antigen is present in the composition in a dose sparing amount.

4. The composition according to claim 1, wherein the TLR2 agonist is selected from the group consisting of Pam₂Cys, Pam₃Cys, Ste₂Cys, Lau₂Cys and Oct₂Cys.

5. The composition according to claim 4, wherein the TLR2 agonist is Pam₂Cys.

6. The composition according to claim 1, wherein the PEG (polyethyleneglycol) has 5 to 22 ethylene oxide subunits.

7. The composition according to claim 6, wherein the PEG (polyethyleneglycol) is (PEG)₁.

8. The composition according to claim 1, wherein the hyper-branched charged peptide comprises R4, R8, K4, K8, E4, E8, D4, D8, H4, or H8.

9. The composition according to claim 1, wherein the TLR targeting moiety is of the formula:

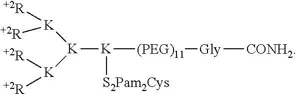

10. The composition according to claim 1, wherein the TLR targeting moiety is of the formula:

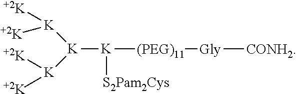

11. The composition according to claim 1, wherein the TLR targeting moiety is of the formula:

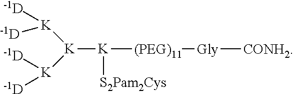

12. The composition according to claim 1, wherein the TLR targeting moiety is of the formula:

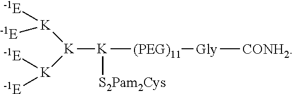

13. The composition according to claim 1, wherein the TLR targeting moiety is of the formula:

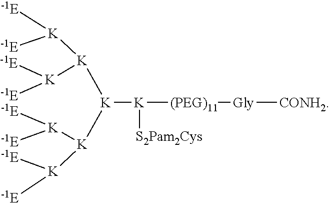

14. The composition according to claim 1, wherein the TLR targeting moiety is of the formula:

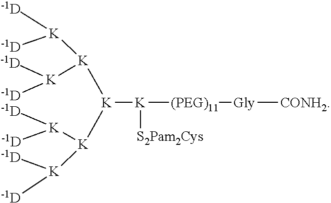

15. The composition according to claim 1, wherein the TLR targeting moiety is of the formula:

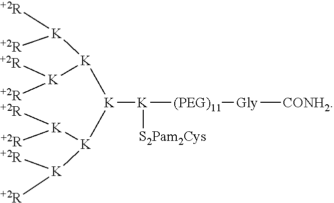

16. The composition according to claim 1, wherein the TLR targeting moiety is of the formula:

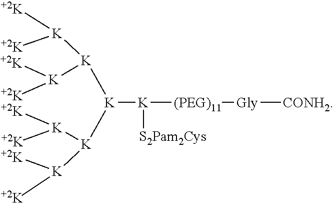

17. The composition according to claim 2, wherein the CTL epitope is derived from a pathogen or a tumour antigen.

18. A method of eliciting a CD8⁻ response in a subject, the method comprising administering to the subject the composition according to claim 1.

19. (canceled)
